(19) United States
(12) Patent Application Publication
(10) Pub. No.: US 2026/0325133 A1
(43) Pub. Date: Oct. 22, 2026

(54) SURGICAL TOOL HAVING AN ARTICULATION LOCK

(71) Applicant: Cilag GmbH International, Zug (CH)

(72) Inventors: Douglas B. Hoffman, Harrison, OH (US); Cory G. Kimball, Hamilton, OH (US); Bradley A. Arnold, Mason, OH (US)

(21) Appl. No.: 19/184,708

(22) Filed: Apr. 21, 2025

(51) Int. Cl. A61B 17/068; A61B 17/00

(52) U.S. Cl. CPC A61B 17/068; A61B 2017/00367; A61B 2017/00477

(57) ABSTRACT

A surgical tool is disclosed. The surgical tool may include a shaft and an articulation lock. The shaft may include an articulation joint. The articulation lock may include a core, a ring, a plurality of wedge locks, and a spring. The plurality of wedge locks may be positioned radially between the core and the ring. The spring may be positioned to bias the plurality of wedge locks toward a locked configuration in which rotation between the core and the ring in a first direction and in a second direction is opposed by the plurality of wedge locks. The first direction may be opposite to the second direction.

### BACKGROUND

This disclosure generally relates to surgical tools and, more particularly, to articulation locks for surgical tools.

A surgical tool may be an articulatable surgical tool having an end effector that is articulatable, or rotatable, relative to a shaft of the surgical tool. The articulatable end effector may be configurable in an orientation to reach tissue that is otherwise inaccessible or difficult to reach with a non-articulatable end effector. An articulatable surgical tool may include an articulation joint or wrist at which the end effector is rotatable relative to the shaft. An articulatable surgical tool may include an articulation lock to hold the end effector at a predefined articulation angle relative to the shaft. Exemplary articulatable surgical tools are described in, for example, U.S. Pat. No. 5,713,505, titled ARTICULATION TRANSMISSION MECHANISM FOR SURGICAL INSTRUMENTS, which issued Feb. 3, 1998; U.S. Pat. No. 9,662,110, titled SURGICAL STAPLING INSTRUMENT WITH AN ARTICULATABLE END EFFECTOR, which issued May 30, 2017; and U.S. Pat. No. 11,234,696, titled ARTICULATION DRIVE FEATURES FOR SURGICAL STAPLER, which issued Feb. 1, 2022.

### SUMMARY

One general aspect includes a surgical tool. The surgical tool may include a shaft and an articulation lock. The shaft may include an articulation joint. The articulation lock may include a core, a ring, a plurality of wedge locks, and a spring. The plurality of wedge locks may be positioned radially between the core and the ring. The spring may be positioned to bias the plurality of wedge locks toward a locked configuration in which rotation between the core and the ring in a first direction and in a second direction is opposed by the plurality of wedge locks. The first direction may be opposite to the second direction.

Implementations of the surgical tool may include one or more of the following features.

The articulation lock may include an inner locking face and an outer locking face, a cavity may be defined between the inner locking face and the outer locking face, and the plurality of wedge locks may be movable along the cavity between the locked configuration and an unlocked configuration. The core may include the inner locking face, and the plurality of wedge locks, in the locked configuration, may be positionable at any location between the inner locking face and the outer locking face. The ring may include the outer locking face, and the plurality of wedge locks, in the locked configuration, may be positionable at any location along the outer locking face. The ring may include a smooth inner perimeter having the outer locking face. The locking configuration may be defined by a contact between the plurality of wedge locks and the outer locking face, and the unlocked configuration may be defined by a separation between the plurality of wedge locks and the outer locking face.

A cavity may be positioned radially between the core and the ring, the cavity may include a first tapered end and a second tapered end, and the plurality of wedge locks may include a first wedge lock movable along the first tapered end and a second wedge lock movable along the second tapered end.

The surgical tool may include an actuator movable along an axis and toward the cavity, the actuator may include a camming finger extending along the axis and toward the cavity, and a translation of the actuator along the axis and toward the cavity may enable the camming finger to cam the plurality of wedge locks out of the locked configuration. The actuator may be rotatable about the axis to drive rotation of the core relative to the outer ring. The surgical tool may include an articulation driver extending through the shaft to the articulation joint, the core may include a driver receivable by the actuator and extending along the axis, the driver may be rotatable about the axis to rotate the core, and the articulation driver may be coupled to the core such that rotation of the core causes translation of the articulation driver. The articulation driver may include a plurality of articulation bands to push and pull a link at the articulation joint. A pin may extend through the core to couple the articulation driver to the core. The spring may include a spring plate, a through slot may be defined in the spring plate and aligned with the cavity to receive the camming finger. The spring plate may include a leaf spring positioned at each end of the through slot and extending into the cavity to bias the plurality of wedge locks towards the locked configuration. The camming finger may include a plurality of chamfers to engage the plurality of wedge locks.

The plurality of wedge locks may include a plurality of wedge lock sets spaced equidistantly around the core, the actuator may include a plurality of camming fingers extending along the axis and toward the cavity, and a translation of the actuator along the axis and toward the cavity may enable the plurality of camming fingers to cam the plurality of wedge locks sets out of the locked configuration.

The surgical tool may include a proximal housing, the shaft may extend distally from the proximal housing, and the articulation lock may be supported by the proximal housing. The proximal housing may include a shaft rotation nozzle, and the articulation lock may be supported by the shaft rotation nozzle.

Another general aspect includes a surgical tool. The surgical tool may include a shaft and an articulation lock. The shaft may include an articulation joint. The articulation lock may include a first body, a second body rotatable about the first body, and a plurality of locks. The plurality of locks may be positioned radially between the first body and the second body. The plurality of locks may be biased toward a locked configuration in which rotation of the second body relative to the first body in a first direction and in a second direction is opposed by the plurality of locks. The first direction may be opposite to the second direction.

Another general aspect includes a surgical tool. The surgical tool may include a shaft and an articulation lock. The shaft may include an articulation joint. The articulation lock may include a central body, a peripheral body, a plurality of rollers, and a spring. The plurality of rollers may be positioned radially between the central body and the peripheral body. The spring may be positioned to bias the plurality of rollers toward a locked configuration in which rotation between the central body and the peripheral body in a first direction and in a second direction is opposed by the plurality of rollers. The first direction may be opposite to the second direction.

### BRIEF DESCRIPTION OF THE DRAWINGS

The accompanying drawings, where like reference numerals may refer to functionally similar elements throughout the separate views, together with the detailed description below, are incorporated in and form part of the specification, and serve to further illustrate aspects of concepts that include the claimed disclosure and explain various principles and advantages of those aspects.

FIG. 1 is an elevation view of a surgical tool foreshortened along an elongated shaft thereof for illustrative purposes, according to at least one aspect of the present disclosure.

FIG. 2 is a plan view of the surgical tool of FIG. 1, according to at least one aspect of the present disclosure.

FIG. 3 is an exploded, perspective view of an articulation assembly, according to at least one aspect of the present disclosure.

FIG. 4A is a plan view of an articulation lock of the articulation assembly of FIG. 3, according to at least one aspect of the present disclosure.

FIG. 4B is a plan view of the articulation lock of FIG. 4A with a spring plate removed for illustrative purposes, according to at least one aspect of the present disclosure.

FIG. 5A is an enlarged plan view of a portion of the articulation lock of FIG. 4B, in which the articulation lock is in a locked configuration, according to at least one aspect of the present disclosure.

FIG. 5B is an enlarged plan view of the portion of the articulation lock of FIG. 4B, in which the articulation lock is in an unlocked configuration, according to at least one aspect of the present disclosure.

FIG. 6 is an elevation view of a portion of the articulation lock of FIG. 4B with the outer ring removed to expose wedge locks in a cavity of the articulation lock and further depicting a camming finger of an actuator, according to at least one aspect of the present disclosure.

The surgical tools and articulation locks disclosed herein have been represented where appropriate by conventional symbols in the drawings, showing only those specific details that are pertinent to understanding the various aspects of the present disclosure so as not to obscure the disclosure with details that will be readily apparent to those of ordinary skill in the art having the benefit of the description herein.

### DETAILED DESCRIPTION

The present disclosure is directed to surgical tools having an articulation lock. In the description, for purposes of explanation and not limitation, specific details are set forth, such as particular aspects, procedures, techniques, etc. to provide a thorough understanding of the present technology. However, it will be apparent to one skilled in the art that the present technology may be practiced in other aspects that depart from these specific details.

Articulatable surgical tools may incorporate different types of locks for holding one or more articulatable components, such as an end effector, for example, in an articulated configuration. For example, the end effector can be locked at a set articulation angle relative to the shaft and the articulation angle can be maintained through a surgical function, such as during a clamping, cutting, and/or stapling motion, for example, until the articulation lock is released. Articulation locks may facilitate controlled positioning of the articulatable surgical tool during a surgical procedure, which may improve a user's ability to manipulate tissue and/or perform other actions with precision and stability, for example.

Articulation locks can include mechanical locks that hold the articulation joint and/or articulation actuator at one of the predefined, indexed positions in which the articulation joint is lockable. For example, engagement between complementary gear-like structures, which may include teeth and corresponding detents, for example, can be structured to hold the articulation joint at one of the predefined, indexed positions within the articulation range of motion. The predefined, indexed positions can be spaced apart at intervals, such as at 15 degree or 45 degree intervals, for example, from an articulated position of the end effector. In certain instances, the teeth of an articulatable surgical instrument (e.g., teeth on an end effector) may engage detents (e.g., detents on the shaft), which form a series of discrete and fixed articulation angles at which the articulatable end effector may be locked.

In certain aspects, an articulation lock may be actuatable to lock an articulation joint at any articulation angle within the articulation range of motion. For example, a wedge-based articulation lock can be structured to lock the articulatable end effector at any desired articulation angle. A wedge-based lock may utilize frictional resistance along a wedged slot or course to lock the articulatable end effector at any desired articulation angle. In such instances, the locked articulation angle is not confined to fixed intervals (such as at a fifteen-degree angle or a 30-degree angle but not therebetween). Infinite locking positions within the articulation range of motion may allow for a smoother and/or more continuous adjustment throughout the range of articulation motion and/or greater flexibility at selecting the desired articulation angle for accessing particular tissue within anatomical and/or surgical constraints, for example.

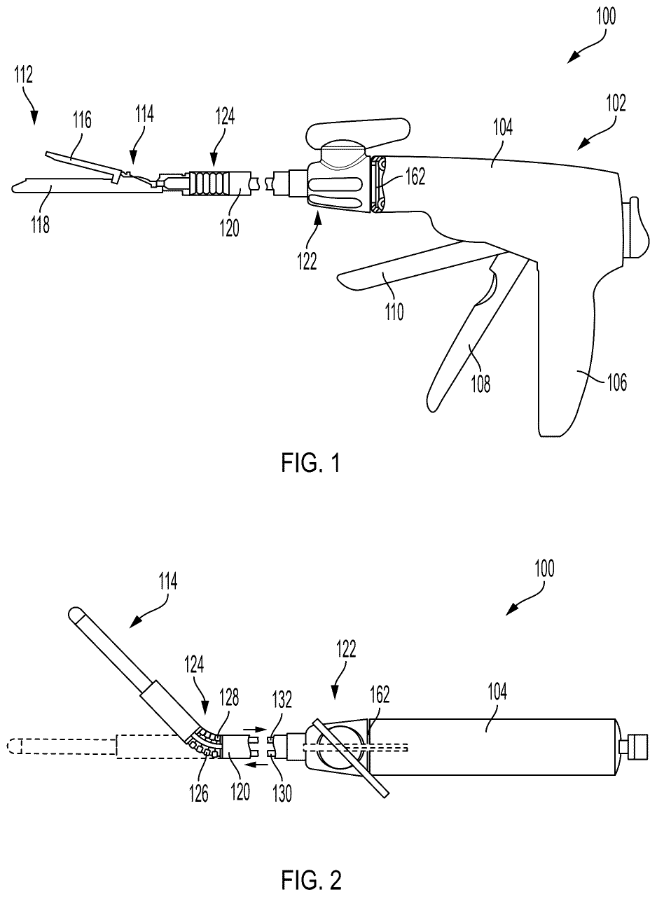

Referring primarily to FIGS. 1 and 2, a surgical tool 100 is disclosed. The surgical tool 100 includes a proximal end 102 having a frame, or handle, 104 adapted to enable a user to grip and manipulate the surgical tool 100. For example, the frame 104 may include a stationary hand grip 106 for placement in the palm of the user's hand, a pivotally-mounted clamping trigger 108 for remotely clamping tissue, and a pivotally-mounted actuating trigger 110 for remotely actuating the surgical tool 100.

The surgical tool 100 further includes a distal end 112 having an end effector 114. The end effector 114 may be a surgical cutting and fastening tool, such as a surgical stapler, for example. More specifically, the end effector 114 includes an elongated anvil 116 and an elongated channel 118 adapted to receive a surgical cartridge which may, for example, have surgical staples removably stored therein. Extending from the frame 104 of the surgical tool 100, and coupling the frame 104 to the end effector 114, is an elongated shaft 120. The surgical tool 100 is exemplary of articulable surgical tools. Similar surgical tools are further disclosed in U.S. Pat. No. 5,713,505, titled ARTICULATION TRANSMISSION MECHANISM FOR SURGICAL INSTRUMENTS, which issued Feb. 3, 1998, and is incorporated by reference herein in its entirety.

It is contemplated that the articulation locks described herein can also be incorporated into non-stapling, articulatable surgical tools, such as surgical graspers, cutters, energy devices (e.g., ultrasonic, radio-frequency (RF), etc.), imaging devices, access devices, and/or fluid delivery and/or extraction devices (e.g., irrigators, smoke evacuators, etc.), for example. Various features of the articulation locks described herein can also be incorporated into robotic surgical tools.

The surgical tool 100 further includes an articulation assembly 122, including an actuator 134, an articulation lock 136, and articulation drivers 130, 132 extending distally to an articulation joint 124 at which the end effector 114 is articulatable relative to the shaft 120. The articulation actuator 134 is positioned at the distal end of the frame 104; however, the articulation actuator 134 can be positioned elsewhere on the surgical tool 100 where it is accessible to a clinician to actuate and control an articulation motion. In other aspects of the present disclosure, the articulation assembly 122 may not include an articulation actuator 134. For example, the articulation joint 124 may be a passive articulation joint, which is actuated by applying a force at the end effector 114, such as by pushing the end effector 114 against patient tissue to articulate the end effector 114 at the joint. Exemplary passive articulation joints are further disclosed in, for example, U.S. Pat. No. 9,662,110, titled SURGICAL STAPLING INSTRUMENT WITH AN ARTICULATABLE END EFFECTOR, which issued May 30, 2017, and is incorporated by reference herein in its entirety.

The articulation lock 136 is also positioned at the distal end of the frame 104. In such instances, the articulation lock 136 may not need to fit within a trocar and, thus, the surgical tool 100 can fit within a smaller diameter opening in the patient, for example. In other instances, the articulation lock 134 can be positioned along the surgical tool 100 intermediate the articulation actuator 134 and the articulation joint 124, such as along the shaft 120 and/or at the articulation joint 124, for example.

Actuation of the articulation assembly 122, is configured to articulate, or rotate, the end effector 114 relative to the elongated shaft 120. In some instances, articulation may be from a non-articulated position to an articulated position, as shown in FIG. 2. However, in other instances, articulation may be from an articulated position to a non-articulated position, or from a first articulated position to a second, different articulated position.

The surgical tool 100 includes an articulation joint 124 intermediate the elongated shaft 120 and the end effector 114. The articulation joint 124 may be a flexible neck or one or more articulation joints, such as a series of articulation joints, for example, to enable articulation of the end effector 114 relative to the elongated shaft 120. The articulation joint 124 may include a first flexible portion, or link, 126 and a second flexible portion, or link, 128. The articulation links 126, 128 can extend through channels in the articulation joint 124. The articulation assembly 122 further includes an articulation driver, which can be a flexible band or cable extending distally from the articulation actuator 122 to the articulation joint 124. For example, the articulation assembly 122 includes a first articulation band 130 coupled to the first link 126, and a second articulation band 132 coupled to the second link 128. Upon actuation of the articulation assembly 122, one of the first and second articulation bands 130, 132 is moved distally while the other of the first and second articulation bands 130, 132 is moved proximally in a push-and-pull manner. In response to the reciprocating movement of the first and second articulation bands 130, 132, the first and second links 126, 128 flex accordingly to provide articulation of the end effector 114 relative to the elongated shaft 120 about an axis and in a first plane.

The surgical tool 100 is articulatable in a single plane (e.g., a transverse plane), which may be referred to as articulation about a yaw axis. In other aspects, the surgical tool 100 can be articulatable in another plane (e.g., a sagittal plane), which may be referred to as articulation about a pitch axis. In still other instances, the surgical tool 100 can be articulatable in multiple planes about orthogonal axes, such as both the yaw axis and the pitch axis, for example. The surgical tool 100 can include a series of articulation joints forming an articulation region and/or at least one biaxial and/or multiaxial joint, for example.

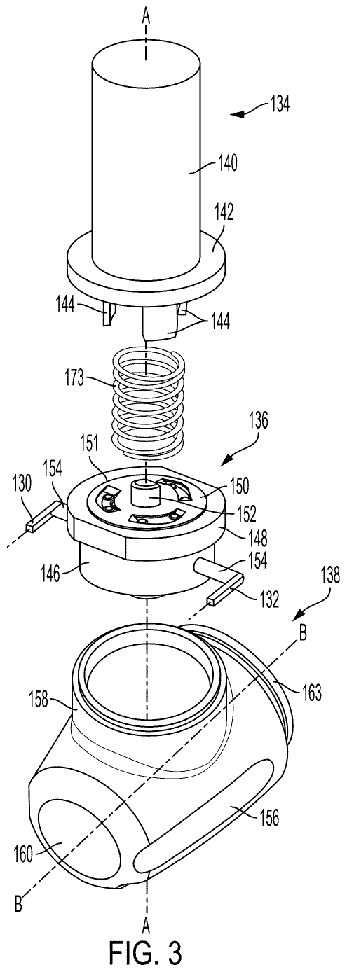

Referring now to FIG. 3, an exploded perspective view of the articulation assembly 122 is disclosed. As shown, the articulation assembly 122 includes the actuator 134 for manual actuation by a clinician, the articulation lock 136, a compression spring 173, and a proximal housing 138, for example. The actuator 134 includes a grip 140, a flange 142, and at least one camming finger 144 extending from the flange 142. Although the grip 140 and the flange 142 are shown to be of cylindrical shape extending along an axis A-A, the grip 140 and the flange 142 may take any suitable shape so long as the actuator 134 may be handled by the clinician and the actuator 134 may engage the articulation lock 136, as further described herein. In some instances, the flange 142 may be omitted and the at least one camming finger 144 may instead extend from the grip 140. Although the actuator 134 is shown to include multiple camming fingers 144 (e.g., three camming fingers) the actuator 134 may include any number of camming fingers, such as, for example, a single camming finger 144. The multiple camming fingers 144 can be spaced equidistant radially from the axis A-A and equidistantly from each other. The number of camming fingers 144 can correspond to the number of wedge lock sets 186, as further disclosed herein.

The articulation lock 136 includes a core 146, an outer ring 148, and a spring 150 in the form of a spring plate 151. In addition to locking the articulation joint 124, the articulation lock 136 is structured to transmit an articulation motion from the actuator 134 to the articulation bands 130, 132. In other words, the articulation lock 136 can form a part of the articulation transmission drive. More specifically, the core 146 includes a drive shaft 152 extending along the axis A-A and adapted to be received by the actuator 134. The core 146 and the drive shaft 152 are rotatable about the axis A-A based on a rotation of the actuator 134 when the drive shaft 152 is received by the actuator 134. Furthermore, the core 146 may include a through hole and a pin 154 extending therethrough. A first end of the pin 154 may be coupled to one of the first and second articulation bands 130, 132 and a second end of the pin 154 may be coupled to the other of the first and second articulation bands 130, 132. As a result, the core 146 may be coupled to the first and second articulation bands 130, 132 such that a rotation of the core 146 and the drive shaft 152 about the axis A-A, based on a rotation of the actuator 134 when the drive shaft 152 is received by the actuator 134, causes one of the first and second articulation bands 130, 132 to move distally and the other of the first and second articulation bands 130, 132 to move proximally in a push-and-pull manner to articulate at least one link of the articulation joint 124, and thus to articulate the end effector 114 relative to the shaft 120.

As described herein, the core 146 is rotatable relative to the outer ring 148, however, in other instances, the outer ring 148 may be rotatable relative to the core 146 to transmit an articulation drive motion. In such instances, the outer ring 148 can include the drive shaft 152 and the pin 154, for example.

Referring still to FIG. 3, the compression spring 173 is positioned along the axis A-A and between the actuator 134 and the articulation lock 136. In various instances, the compression spring 173 can interface with a surface of the flange 142 and a surface of the outer ring 148 or the spring plate 151. In other instances, the flange 142 and/or the outer ring 148 or the spring plate 151 can include a channel to receive a portion of the compression spring 173. In instances where the flange 142 is omitted, the compression spring 173 can instead interface with an interior surface of the grip 140, or a channel therein, for example.

The compression spring 173 is positioned to the actuator 134 along the axis A-A away from the articulation lock 136 into a non-actuated configuration in which the actuator 134 is spaced apart from the articulation lock 136. In the non-actuated configuration, the actuator 134 and, specifically, the at least one camming finger 144 thereof is not positioned to cam the wedge locks 166, 168 into an unlocked configuration 182, as further described herein. The compression spring 173 and, specifically, the biasing force provided by the compression spring 173 can bias the actuator 134 into the non-actuated configuration until the spring force is overcome, such as by a clinician actuating the actuator 134 to compress the compression spring 173 to unlock the articulation lock 136 and articulate the end effector 114.

As further described herein, the unlocked configuration 182 corresponds to the actuator 134 and, specifically, one of the camming fingers 144 thereof having been received within a through slot 162 of the spring plate 151 and a cavity 164 positioned radially between the core 146 and the outer ring 148. In various instances, the actuator 134 having been received within the through slot 162 and the cavity 164 is a result of a clinician applying a force to the actuator 134 along the axis A-A and toward the articulation lock 136. In various instances, to actuate the articulation lock 136, the force applied by the clinician to the actuator 134 along the axis A-A and toward the articulation lock 136 must be sufficient to overcome the biasing force provided to the actuator 134 by the compression spring 173 along the axis A-A and away from the articulation lock 136.

Although the present disclosure has been described with respect to the compression spring 173, alternative springs and resilient and/or biasing members are contemplated. The biasing member can be selected and positioned to bias the actuator 134 into the non-actuated configuration in which the at least one camming finger 144 does not cam the wedge locks 166, 168 into the unlocked configuration 182. For example, alternative biasing members for the compression spring 173 can include torsion springs or any suitable spring that can be selected and positioned to bias the actuator 134 toward the non-actuated configuration.

Referring still to FIG. 3, the proximal housing 138 may include a shaft rotation nozzle 156, an articulation lock housing 158, an axial bore 160, and a groove 163. The shaft rotation nozzle 156 may be coupled to the elongated shaft 120 such that a rotation of the shaft rotation nozzle 156 about an axis B-B, which corresponds to the longitudinal axis of the elongate shaft 120 in FIG. 3, for example, causes the elongated shaft 120 and the end effector 114 to rotate about the axis B-B. The articulation lock housing 158 is dimensioned and structured to receive and house the articulation lock 136, and the axial bore 160 is dimensioned and structured to receive a proximal portion of the elongated shaft 120 and the first and second articulation bands 130, 132. Furthermore, the groove 163 is configured to rotationally couple the proximal housing 138 to the frame 104 as shown in FIGS. 1 and 2, for example.

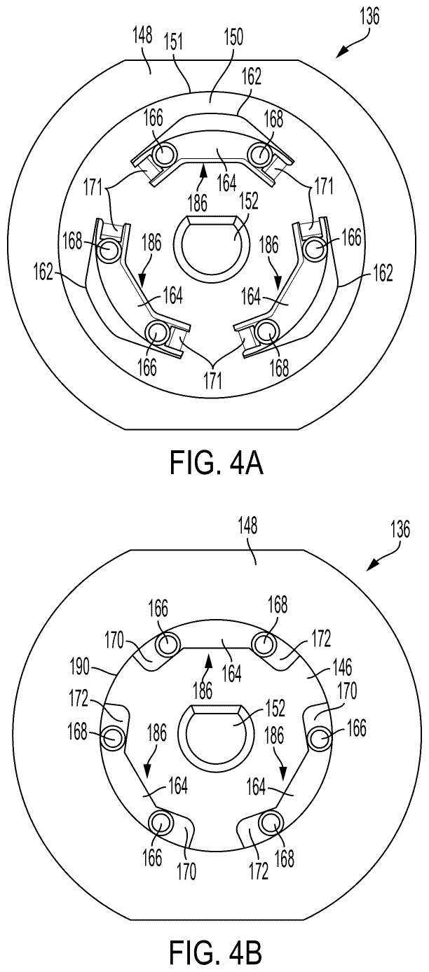

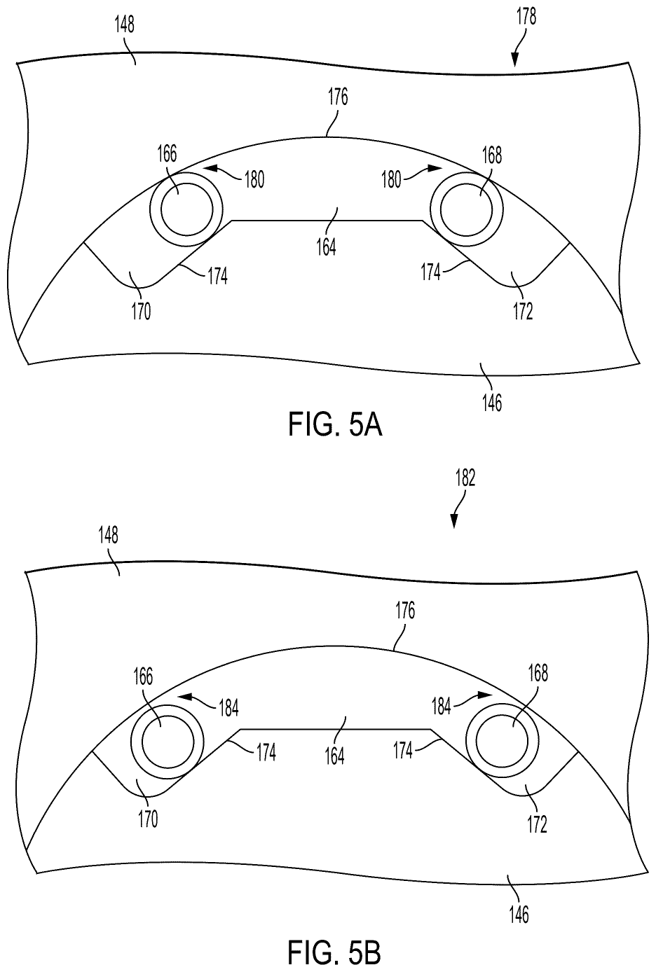

Referring now to FIGS. 4A-6, the articulation lock 136 is actuatable to move between a locked configuration 178 (FIGS. 4A, 4B, 5A, and 6), in which articulation of the end effector 114 relative to the shaft 120 is prevented or restrained, and an unlocked configuration 182 (FIG. 5B), in which articulation of the end effector 114 relative to the shaft 120 within the limits of the articulation joint 124 is permitted by the articulation lock 136. As shown in FIG. 4A, and as previously described herein, the spring 150 and, specifically, the spring plate 151, includes a through slot 162 aligned with a space or cavity 164 defined radially between the core 146 and the outer ring 148. Wedge locks 166, 168, which may also be referred to as wedge rollers, are positioned within the cavity 164 and biased toward the locked configuration 178 by the spring 150. A pair, or set, of wedge locks 166, 168 form a wedge lock set 186, for example. The spring plate 151 includes a leaf spring 171 positioned at each end of the through slot 162. The leaf springs 171 extend into the cavity 164 to interface with the respective wedge lock 166, 168 adjacent thereto. As shown by FIG. 4B, which illustrates the articulation lock 136 of FIG. 4A without the spring plate 151 for illustrative purposes, the wedge locks 166, 168 are positioned within and movable along at least a portion of the cavity 164. For example, the wedge lock 166 is movable along a first tapered end 170 of the cavity 164, and the wedge lock 168 is movable along a second tapered end 172 of the cavity 164.

In various aspects, the outer ring 148 has an inner perimeter 190 that is smooth. For example, the inner perimeter 190 may have a constant curvature. The core 146 is dimensioned to fit within the inner perimeter 190 and to rotate along the inner perimeter 190. For example, an outer boundary of the core 146 defines a complementary curvature to the inner perimeter 190 such that the outer boundary of the core 146 rotates within the inner perimeter 190 of the outer ring 148.

The core 146 further includes perimeter surfaces positioned radially inward from the radially outermost surface of the core 146 and from the inner perimeter 190 of the outer ring 148 to form the cavity 164 between the outer ring 148 and the core 146, which is dimensioned to accommodate the wedge locks 166, 168 and the camming finger 144. The perimeter surfaces of the core 146 that form and define a portion of the cavity 164 are not equidistantly spaced from the inner perimeter 190 of the outer ring 148. For example, the perimeter surfaces of the core 146 forming the cavity 164 are angled or sloped to change the width of the cavity 164 along the length thereof.

Referring primarily to FIGS. 5A and 5B, the cavity 164 defines an arced profile having a constant-radius outer surface (e.g., a circular arc corresponding to the curvature of the inner perimeter 190 of the outer ring 148) forming an outer locking face 176 of the articulation lock 136. The cavity 164 is further defined by angled inner surfaces of the core 146 forming an inner locking face 174 for each wedge lock 166, 168. The cavity 164 includes a central opening (to receive the camming finger 144, for example) and tapered ends 170, 172 on opposing side of the central opening. The tapered ends 170, 172 taper or narrow from the end of the cavity inward toward the central opening. The diameter of the wedge locks 166, 168 is greater than the narrowest width of the cavity 164 along the tapered ends 170, 172 such that the wedge locks 166, 168 are prevented from moving into the central opening. The wedge locks 166, 168 can be free to move within the wider portions of the cavity 164 under the biasing forces of the spring 150 and the camming forces of the camming finger 144, for example.

Referring primarily now to FIG. 5A, the locked configuration 178 of the articulation lock 136 is shown. Although not shown in FIG. 5A, the spring 150 and, specifically, the leaf spring 171 positioned at each end of the through slot 162 of the spring plate 151 is positioned to bias the respective wedge lock 166, 168 into the locked configuration 178. For example, the leaf springs 171 are positioned to bias the wedge locks 166, 168 of a wedge lock set 186 towards each other, the central opening, and a centerline, or plane of symmetry, spaced between the wedge locks 166, 168 and including the axis A-A. The wedge lock set 186 can be symmetrical about the plane of symmetry, for example.

In the locked configuration 178, each respective wedge lock 166, 168 is engaged with the inner locking face 174 of the core 146 and with the outer locking face 176 of the outer ring 148 at an interference point, or contact, 180. Friction between the wedge locks 166, 168 and the locking faces 174, 176 prevents rotation of the core 146 relative to the outer ring 148. Moreover, the tapered or narrowed geometry along the tapered ends 170, 172 of the cavity 164 blocks further motion of the wedge locks 166, 168 toward each other along the tapering or narrowing length of the tapered ends 170, 172 to hold and maintain the frictional engagement between the wedge locks 166, 168 and the locking faces 174, 176.

In various aspects, due to the bias of the wedge locks 166, 168 between the inner locking face 174 and the outer locking face 176 by the leaf springs 171 of the spring plate 151, rotation between the core 146 and the outer ring 148 in a first direction (e.g., counterclockwise) and in a second direction (e.g., clockwise) is opposed. For example, the contact 180 between the wedge lock 166 and the border of the cavity 164 at the first tapered end 170 can prevent rotation of the core 146 in the first direction (e.g., counterclockwise), and the contact 180 between the wedge lock 168 and the border of the cavity 164 of the second tapered end 172 can prevent rotation of the core 146 in the second direction (e.g., clockwise). As a result, the articulation lock 136 is in the locked configuration 178 and the end effector 114 and the articulation joint 124 are locked in their respective positions.

In various aspects, the wedge-shaped geometry of the tapered ends 170, 172 provides a robust frictional lock. Forces attempting to overcome the articulation lock can further wedge the wedge locks 166, 168 along the tapered ends 170, 172 to further secure the position of the articulation joint. Moreover, the tapered ends 170, 172 of the cavity 164, which taper in opposite directions, i.e., towards each other, and become narrower towards the centerline of the articulation lock 136, cooperate with the wedge locks 166, 168 to provide bidirectional resistance to rotation.

In various aspects, the tapering or slope of the tapered ends 170, 172 can be minimalized within the footprint of the articulation lock 136. Minimizing the slope can direct the majority of the load between the wedge locks 166, 168 and the locking faces 174, 176 in a radial direction, for example. For example, the slope can be less than 20 degrees and, in various aspects, about 15 degrees, for example. In other instances, the slope can be less than 15 degrees or less than 10 degrees and, in still other instances, can be about 20 degrees or more than 20 degrees, for example.

In various instances, the wedge locks 166, 168, the inner locking face 174, and the outer locking face 176 can have a similar hardness value, which may be selected to resist uneven frictional wear between the components. The wedge locks 166, 168, the inner locking face 174, and the outer locking face 176 can be comprised of the same material, which may be a metal, for example.

Referring now to FIG. 5B, the unlocked configuration 182 of the articulation lock 136 is shown. Although not shown in FIG. 5B for illustrative purposes, the actuator 134 and, specifically, one of the camming fingers 144 thereof is received within the through slot 162 of the spring plate 151 and the cavity 164 positioned radially between the core 146 and the outer ring 148. As a result, the camming finger 144 is selectively positionable to cam the wedge locks 166, 168 away from each other, out of the locked configuration 178, and into an unlocked configuration 182, which may be defined by a separation or gap 184 between the wedge locks 166, 168 and the outer locking face 176 and/or by a separation or gap 184 between the wedge locks 166, 168 and the inner locking face 174. Due to the bias of the wedge locks 166, 168 out of the locked configuration 178 and into the unlocked configuration 182, rotation between the core 146 and the outer ring 148 in a first direction (e.g., counterclockwise) and in a second direction (e.g., clockwise) is no longer opposed. Accordingly, the articulation lock 136 is in an unlocked configuration 182 and the end effector 114 and the articulation joint 124 are free to articulate according to the articulation transmission drive further described herein.

In various instances, upon reaching the desired articulation angle, the articulation lock 136 is actuatable to hold the end effector 114 in the desired articulation angle. The desired articulation angle can be any angle within the articulation range of motion. Infinite locked positions for an articulation angle are achieved with the articulation lock 136 and wedge locks 166, 168 thereof, which are biased into frictional engagement with the inner and outer locking faces 174, 176 by the spring 150 at any articulation angle of the end effector 114. For example, the outer ring 148 is configured to rotate about the core 146 to push and pull the articulation bands 130, 132 and effect an articulation motion of the end effector 114. Upon reaching the desired articulation angle of the end effector 114, corresponding to a specific angular position of the outer ring 148, the articulation lock 136 can be actuated to fix the angular position of the outer ring 148 relative to the core 146, which corresponds to locking or holding the articulation angle of the end effector 114. The wedged geometry at both ends of the slot 164, i.e., the tapered ends 170, 172, prevents rotation of the outer ring 148 in both directions (e.g., counterclockwise and clockwise) relative to the core 146.

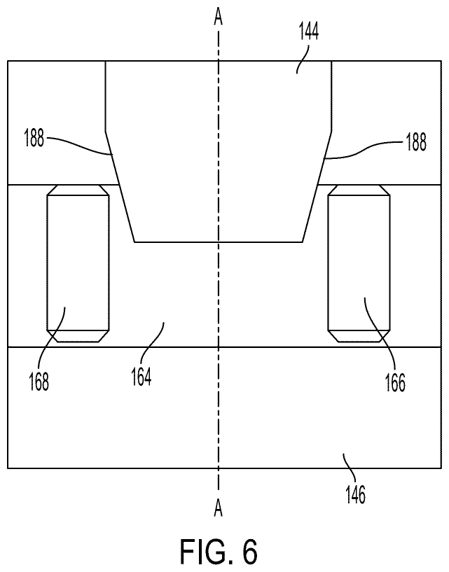

Referring now to FIG. 6, a portion of the articulation lock 136 and the camming finger 144 of the actuator 134 is shown. The wedge locks 166, 168 are positioned within the cavity 164 defined between the core 146 and the outer ring 148 (FIGS. 4A-5B). As the actuator 134, and thus the camming finger 144 thereof, is moved along the axis A-A and toward the cavity 164, a chamfer 188 on each side of the camming finger 144 is positioned to engage the corresponding wedge locks 166, 168 to cam the wedge locks 166, 168 out of the locked configuration 178 and into the locked configuration 182. As the actuator 134, and thus the camming finger 144, is moved along the axis A-A and away from the cavity 164, the chamfers 188 of the camming finger 144 no longer engage the wedge locks 166, 168, and thus the spring 150 and, specifically, the leaf spring 171 positioned at each end of the through slot 162 of the spring plate 151, is positioned to bias the wedge locks 166, 168 back into the locked configuration 178. As a result, rotation between the core 146 and the outer ring 148 in a first direction (e.g., counterclockwise) and in a second direction (e.g., clockwise) is opposed, and the articulation lock 136 is in the locked configuration 178, thereby preventing articulation at the articulation joint 124 and, thus, rotation of the end effector 114 relative to the shaft 120.

Although a single wedge lock set 186 is described herein with respect to FIGS. 5A, 5B, and 6, the articulation lock 136 may include multiple wedge lock sets 186 spaced equidistantly apart around the core 146, each comprising the wedge locks 166 and 168, as shown in FIG. 3, for example. Furthermore, the actuator 134 may include multiple camming fingers 144, where each camming finger 144 is aligned with a wedge lock set 186 and adapted to cam the wedge locks 166, 168 thereof out of the locked configuration 178 and into the unlocked configuration 182. In various instances, the number and position of the camming fingers 144 corresponds to the number and position of wedge lock sets 186. The camming fingers 144 may simultaneously engage the wedge lock sets 186 to simultaneously release the wedge locks 166, 168 thereof.

Additionally, although the present disclosure has been described with respect to the spring 150 and, specifically, the spring plate 151 including the leaf spring 171 positioned at each end of the through slot 162, alternative resilient and/or biasing members are contemplated. The biasing member can be selected and positioned to bias the wedge locks 166, 168 into the locked configuration 178. For example, alternative biasing members for the spring 150 and, specifically, the spring plate 151 including the leaf spring 171 can include coil springs, compression springs, and/or magnets, which can be retained in the through slot 162 to bias the wedge locks 166, 168 toward the locked configuration 178.

Examples of the devices, systems, and methods disclosed herein are provided in the following numbered clauses. An aspect of the devices, systems, and methods may include any one, more than one, and any combination of the numbered clauses below:
Clause 1: A surgical tool, comprising: a shaft comprising an articulation joint; and an articulation lock, comprising: a core; a ring; a plurality of wedge locks positioned radially between the core and the ring; and a spring positioned to bias the plurality of wedge locks toward a locked configuration in which rotation between the core and the ring in a first direction and in a second direction is opposed by the plurality of wedge locks, wherein the first direction is opposite to the second direction.
Clause 2: The surgical tool of Clause 1, wherein the articulation lock further comprises an inner locking face and an outer locking face, wherein a cavity is defined between the inner locking face and the outer locking face, and wherein the plurality of wedge locks are moveable along the cavity between the locked configuration and an unlocked configuration.
Clause 3: The surgical tool of Clause 2, wherein the plurality of wedge locks, in the locked configuration, are positionable in the cavity at any location along the outer locking face.
Clause 4: The surgical tool of Clause 2, wherein the core comprises the inner locking face, and wherein the ring comprises the outer locking face.
Clause 5: The surgical tool of Clause 2, wherein the ring comprises a smooth inner perimeter comprising the outer locking face.
Clause 6: The surgical tool of Clause 2, wherein the locked configuration is defined by a contact between the plurality of wedge locks and the outer locking face, and wherein the unlocked configuration is defined by a gap between the plurality of wedge locks and at least one of the inner locking face or the outer locking face.
Clause 7: The surgical tool of Clause 1, wherein a cavity is positioned radially between the core and the ring, wherein the cavity comprises a first tapered end and a second tapered end, and wherein the plurality of wedge locks comprises a first wedge lock movable along the first tapered end and a second wedge lock movable along the second tapered end.
Clause 8: The surgical tool of Clause 7, further comprising an actuator movable along an axis and toward the cavity, wherein the actuator comprises a camming finger extending along the axis and toward the cavity, and wherein a translation of the actuator along the axis and toward the cavity enables the camming finger to cam the plurality of wedge locks out of the locked configuration.
Clause 9: The surgical tool of Clause 8, wherein the actuator is rotatable about the axis to drive rotation of the core relative to the outer ring.
Clause 10: The surgical tool of Clause 8, further comprising an articulation driver extending through the shaft to the articulation joint, wherein the core further comprises a drive shaft receivable by the actuator and extending along the axis, wherein the drive shaft is rotatable about the axis to rotate the core, and wherein the articulation driver is coupled to the core such that rotation of the core causes translation of the articulation driver.
Clause 11: The surgical tool of Clause 10, wherein the articulation driver comprises a plurality of articulation bands to push and pull a link at the articulation joint.
Clause 12: The surgical tool of Clause 10, wherein a pin extending through the core couples the articulation driver to the core.
Clause 13: The surgical tool of Clause 8, wherein the spring comprises a spring plate, and wherein a through slot is defined in the spring plate and aligned with the cavity to receive the camming finger.
Clause 14: The surgical tool of Clause 13, wherein the spring plate comprises a leaf spring positioned at each end of the through slot and extending into the cavity to bias the plurality of wedge locks toward the locked configuration.
Clause 15: The surgical tool of Clause 8, wherein the camming finger comprises a plurality of chamfers to engage the plurality of wedge locks.
Clause 16: The surgical tool of Clause 8, wherein the plurality of wedge locks comprises a plurality of wedge lock sets spaced equidistantly around the core, wherein the actuator comprises a plurality of camming fingers extending along the axis and toward the cavity, and wherein a translation of the actuator along the axis and toward the cavity enables the plurality of camming fingers to cam the plurality of wedge lock sets out of the locked configuration.
Clause 17: The surgical tool of Clause 1, further comprising a proximal housing, wherein the shaft extends distally from the proximal housing, and wherein the articulation lock is supported by the proximal housing.
Clause 18: The surgical tool of Clause 17, wherein the proximal housing comprises a shaft rotation nozzle, and wherein the articulation lock is supported by the shaft rotation nozzle.
Clause 19: A surgical tool, comprising: a shaft comprising an articulation joint; and an articulation lock, comprising: a first body; a second body rotatable about the first body; a plurality of locks positioned radially between the first body and the second body, wherein the plurality of locks are biased toward a locked configuration in which rotation of the second body relative to the first body in a first direction and in a second direction is opposed by the plurality of locks, and wherein the first direction is opposite to the second direction.
Clause 20: A surgical tool, comprising: a shaft comprising an articulation joint; and an articulation lock, comprising: a central body; a peripheral body; a plurality of rollers positioned radially between the central body and the peripheral body; and a spring positioned to bias the plurality of rollers toward a locked configuration in which rotation between the central body and the peripheral body in a first direction and in a second direction is opposed by the plurality of rollers, and wherein the first direction is opposite to the second direction.

The terms “proximal” and “distal” are used herein with reference to a clinician manipulating a surgical device or, in the case or a robotic tool, to a robot manipulating the tool. The term “proximal” refers to the portion closest to the clinician or the robot (e.g., the housing) and the term “distal” refers to the portion located away from the clinician or robot (e.g., the end effector). It will be further appreciated that, for convenience and clarity, spatial terms such as “vertical”, “horizontal”, “up”, “down”, “clockwise”, and “counterclockwise” may be used herein with respect to the drawings. However, surgical devices are used in many orientations and positions, and these terms are not intended to be limiting and/or absolute.

The singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance (e.g., +20%) that allows the part or collection of components to function for its intended purpose as described herein.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.” With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-15. (canceled)

16. A surgical tool, comprising:
a shaft comprising an articulation joint; and
an articulation lock, comprising:
a core;
a ring;
a plurality of wedge locks positioned radially between the core and the ring; and
a spring positioned to bias the plurality of wedge locks toward a locked configuration in which rotation between the core and the ring in a first direction and in a second direction is opposed by the plurality of wedge locks, wherein the first direction is opposite to the second direction; and
wherein the articulation lock further comprises an inner locking face and an outer locking face, wherein a cavity is defined between the inner locking face and the outer locking face, and wherein the plurality of wedge locks are moveable along the cavity between the locked configuration and an unlocked configuration.

17. (canceled)

18. The surgical tool of claim 16, wherein the plurality of wedge locks, in the locked configuration, are positionable in the cavity at any location along the outer locking face.

19. The surgical tool of claim 16, wherein the core comprises the inner locking face, and wherein the ring comprises the outer locking face.

20. The surgical tool of claim 16, wherein the ring comprises a smooth inner perimeter comprising the outer locking face.

21. The surgical tool of claim 16, wherein the locked configuration is defined by a contact between the plurality of wedge locks and the outer locking face, and wherein the unlocked configuration is defined by a gap between the plurality of wedge locks and at least one of the inner locking face or the outer locking face.

22. The surgical tool of claim 16, wherein a cavity is positioned radially between the core and the ring, wherein the cavity comprises a first tapered end and a second tapered end, and wherein the plurality of wedge locks comprises a first wedge lock movable along the first tapered end and a second wedge lock movable along the second tapered end.

23. The surgical tool of claim 22, further comprising an actuator movable along an axis and toward the cavity, wherein the actuator comprises a camming finger extending along the axis and toward the cavity, and wherein a translation of the actuator along the axis and toward the cavity enables the camming finger to cam the plurality of wedge locks out of the locked configuration.

24. The surgical tool of claim 23, wherein the actuator is rotatable about the axis to drive rotation of the core relative to the ring.

25. The surgical tool of claim 23, further comprising an articulation driver extending through the shaft to the articulation joint, wherein the core further comprises a drive shaft receivable by the actuator and extending along the axis, wherein the drive shaft is rotatable about the axis to rotate the core, and wherein the articulation driver is coupled to the core such that rotation of the core causes translation of the articulation driver.

26. The surgical tool of claim 25, wherein the articulation driver comprises a plurality of articulation bands to push and pull a link at the articulation joint.

27. The surgical tool of claim 25, wherein a pin extending through the core couples the articulation driver to the core.

28. The surgical tool of claim 23, wherein the spring comprises a spring plate, and wherein a through slot is defined in the spring plate and aligned with the cavity to receive the camming finger.

29. The surgical tool of claim 28, wherein the spring plate comprises a leaf spring positioned at each end of the through slot and extending into the cavity to bias the plurality of wedge locks toward the locked configuration.

30. The surgical tool of claim 23, wherein the camming finger comprises a plurality of chamfers to engage the plurality of wedge locks.

31. The surgical tool of claim 23, wherein the plurality of wedge locks comprises a plurality of wedge lock sets spaced equidistantly around the core, wherein the actuator comprises a plurality of camming fingers extending along the axis and toward the cavity, and wherein a translation of the actuator along the axis and toward the cavity enables the plurality of camming fingers to cam the plurality of wedge lock sets out of the locked configuration.

32. The surgical tool of claim 16, further comprising a proximal housing, wherein the shaft extends distally from the proximal housing, and wherein the articulation lock is supported by the proximal housing.

33. The surgical tool of claim 32, wherein the proximal housing comprises a shaft rotation nozzle, and wherein the articulation lock is supported by the shaft rotation nozzle.

34. A surgical tool, comprising:
a shaft comprising an articulation joint; and
an articulation lock, comprising:
a core;
a ring rotatable about the core; and
a plurality of locks positioned radially between the core and the ring, wherein the plurality of locks are biased toward a locked configuration in which rotation of the ring relative to the core in a first direction and in a second direction is opposed by the plurality of locks, and wherein the first direction is opposite to the second direction; and
wherein the articulation lock further comprises an inner locking face and an outer locking face, wherein a cavity is defined between the inner locking face and the outer locking face, and wherein the plurality of locks are moveable along the cavity between the locked configuration and an unlocked configuration.

35. A surgical tool, comprising:
a shaft comprising an articulation joint; and
an articulation lock, comprising:
a core;
a ring;
a plurality of rollers positioned radially between the central body and the peripheral body; and
a spring positioned to bias the plurality of rollers toward a locked configuration in which rotation between the core and the ring in a first direction and in a second direction is opposed by the plurality of rollers, and wherein the first direction is opposite to the second direction; and
wherein the articulation lock further comprises an inner locking face and an outer locking face, wherein a cavity is defined between the inner locking face and the outer locking face, and wherein the plurality of rollers are moveable along the cavity between the locked configuration and an unlocked configuration.

36. The surgical tool of claim 16, wherein the ring comprises an inner perimeter and an outer perimeter and wherein the inner perimeter has a constant curvature.

37. The surgical tool of claim 16, wherein the ring comprises an inner perimeter and an outer perimeter and wherein the inner perimeter defines an arced profile.
